(19) United States
(12) Patent Application Publication
(10) Pub. No.: US 2026/0356037 A1
(43) Pub. Date: Nov. 12, 2026

(54) PRINTING OBJECTS FROM A WELL

(71) Applicant: Poietis Biosystems, Pressac (FR)

(72) Inventors: Antonio Iazzolino, Pessac (FR); Bertrand Viellerobe, Merignac (FR); Fabien Guillemot, Preignac (FR)

(21) Appl. No.: 19/120,967

(22) PCT Filed: Oct. 20, 2023
(86) PCT No.: PCT/EP2023/079253

§ 371 Date: Apr. 14, 2025

(30) Foreign Application Priority Data
Oct. 20, 2022 (FR) 2210891

(51) Int. Cl. B29C 64/223; B29C 64/106; B29C 64/245; B29C 64/268; B33Y 10/00; B33Y 30/00

(52) U.S. Cl. CPC B29C 64/223; B29C 64/106; B29C 64/245; B29C 64/268; B33Y 10/00; B33Y 30/00

(57) ABSTRACT

A method for transferring an object from a donor substrate toward a target substrate the object having dimensions (Dₓ, D_{y}, D_{z}), the transfer being ensured by local energy excitation of the liquid to form a local cavitation bubble at the object, wherein: a. the donor substrate includes a micro-well plate having a plurality of tapered wells, a width (L) of the base of the wells being L>2DₓD_{y} and a height (h) of the well being h>2D_{z}; and b. the transfer takes place at a first energy level if the ratio of the dimension (D_{z}) of the object to the thickness (e) of the liquid film is less than 1, and at a higher second energy level if the ratio is greater than 1. A system may be used to perform such a method.

### CROSS-REFERENCE TO RELATED APPLICATIONS

This application is a national phase entry under 35 U.S.C. § 371 of International Patent Application PCT/EP2023/079253, filed Oct. 20, 2023, designating the United States of America and published as International Patent Publication WO 2024/084031 A1 on Apr. 25, 2024, which claims the benefit under Article 8 of the Patent Cooperation Treaty of French Patent Application Serial No. FR2210891, filed Oct. 20, 2022.

### TECHNICAL FIELD

The present disclosure relates to the field of additive manufacturing of a material by repeated transfers of particles between a donor substrate and a receiver substrate, a film of carrier liquid containing the particles to be transferred being deposited on the donor substrate, and, in particular, to the field of bioprinting. The specific field of bioprinting concerns the use of digital manufacturing processes to organize and assemble biological tissue components in 2D and 3D, with the aim of producing grafts for regenerative medicine or physiological models for biomedical and pharmaceutical research.

### BACKGROUND

The general principle is to transfer biological (e.g., cells), organic or mineral objects from a carrier liquid deposited on a substrate in the form of a film, and to deliver an energy pulse to form a cavitation bubble, either directly by vaporization of the liquid part in the focal field of the energy source—usually a laser—or via a thin metal coating forming a sacrificial layer on the donor substrate. This cavitation bubble carries the particle(s) in the firing axis toward a receiving substrate, on which the transferred particles accumulate as the shots are repeated.

In the state of the art, Chinese Patent Publication CN115198376 is known, which relates to a micropore array chip and a single-cell sorting method for laser-induced direct transfer, a metal-coated glass sheet being coated with a of microporous membrane layer; the microporous membrane is a biocompatible film and is coated onto the metal-coated glass sheet by adopting micro-nano processing technology; the single cell forms a single-cell micro-liquid drop due to the hydrophobic characteristic of the micropore array chip material, and the single-cell micro-liquid drop is fixed in the micropore so that single-cell capture is achieved and subsequent single-cell sorting is facilitated.

U.S. Patent Application Publication No. US2020009877 proposes a solution for depositing particles on a target from a transparent slide having a film formed by a fluid containing particles in suspension, by locally exciting the film with a laser; it includes means for observing the local excitation region. The observation means comprise a sensor and a light source whose optical axes are substantially shared in a space between an optical splitter and that of the film. The optical beam of the imaging system and the optical beam of the laser are coaxial in the part between the controlled optical deflection means and the film. The device comprises a first focusing optical unit arranged between the controlled optical deflection means and the film. The device comprises a second optical image-combining unit positioned between the sensor and the separator, with the sensor positioned in the focal plane of the second optical unit.

Chinese Patent Publication CN113021874 describes a single-cell printing method based on transfer induced by an annular laser spot, which includes the following steps:
step 1, after pulsed laser beams that are uniformly distributed are incident on an element of the annular light spot forming system, the emerging light is focused to form an annular light spot on a sacrificial layer through a transparent constraint layer;
step 2, the sacrificial layer is ablated by laser light and heat to form an annular plasma cavitation bubble;
step 3, rapid expansion of the annular cavitation bubbles and removal of the cell solution outside the rings, and parallel downward thrust of the target transfer cells in the center of the rings under the expansion of the cavitation bubbles;
step 4, collapse of the annular cavitation bubbles and complete expulsion of the target transfer cells by collapse of the shock waves generated in the direction vertical to the central position of the ring;
step 5, under the action of gravity and momentum, print the target transfer cells and the transported solution onto a receiver plate.

U.S. Patent Application Publication No. US2017225390 concerns a method for additive manufacturing of a three-dimensional object. The method involves sequentially forming a plurality of layers, each of which is structured according to the shape of a cross-section of the object. In some embodiments, forming at least one of the layers comprises performing a raster scan to distribute at least a first building material composition, and a vector scan to distribute at least a second building material composition. Vector scanning optionally takes place along a path selected to form at least one structure selected from the group consisting of (i) an elongated structure, (ii) a boundary structure at least partially surrounding a region filled with the first building material, and (iii) an inter-layer connecting structure.

U.S. Patent Application Publication No. US2016259250 relates to a method of providing a patterned structure on a substrate, the method comprising:
providing a donor substrate disposed between a light source and an acceptor substrate, the donor substrate comprising a donor material;
providing a mask disposed between the light source and the donor substrate, the mask comprising a mask pattern for shaping light from the light source impinging on the donor substrate, the shaped light corresponding to the patterned structure to be created, the shaped light impinging on the donor substrate causing the donor material to be released from the donor substrate and transferred to the acceptor substrate to form the patterned structure thereon; wherein;
the structured light is split into a plurality of separate beams of homogeneous size impinging simultaneously on the donor substrate to cause the donor material to be released from the donor substrate in the form of separate droplets of homogeneous size.

U.S. Patent Application Publication No. US2018090314 relates to a material disposition method consisting of positioning a donor film comprising a donor material at a predefined distance from an acceptor substrate, with the donor film facing the acceptor substrate. One or more pulses of laser radiation are directed onto the donor film at a given location to induce the formation of a protrusion made of the donor material. A distal tip of the protrusion touches the acceptor substrate and is deposited on it, while the protrusion is still in contact with the donor film. A spot of donor material is formed on the acceptor substrate by increasing the separation between donor film and acceptor substrate so as to detach the distal tip from the donor film protrusion.

Prior art solutions are poorly suited to particle sizes in excess of 100 μm, and also pose difficulties in terms of centering the shot with respect to the particle to be transferred.

Furthermore, prior art solutions involve object handling operations that present risks of loss of viability, particularly for fragile biological objects.

The present disclosure aims to solve the problem of prior art solutions to improve the quality and accuracy of transferring objects to the receiving substrate when the objects to be transferred are of different sizes.

### BRIEF SUMMARY

In order to overcome these drawbacks, the present disclosure relates in its most general sense to a method for manufacturing a material by transferring at least one particle from a donor substrate toward a target substrate. More particularly, it concerns a method for transferring an object from a donor substrate toward a target substrate, a carrier liquid film containing the particles to be transferred being deposited on the donor substrate containing the objects to be transferred of dimensions (Dₓ, D_{y}, D_{z}), the transfer being effected by local energy excitation to form a cavitation bubble localized at an object, wherein:
a. the substrate consists of a micro-well plate having a plurality of wells each forming a receptacle having an open upper base that tapers toward the bottom, the width (L) of the base of the wells being L>2DₓD_{y} and the height (h) of the well h>2D_{z}, and
b. the transfer takes place
   according to a first transfer mode with a first energy level E₁, if the ratio of the dimension (D_{z}) of the object to the thickness (e) of the liquid film is less than 1,
   according to a second transfer mode with a second energy level E₂>E₁ if the ratio of the dimension (D_{z}) of the object to the thickness (e) of the liquid film is greater than 1.

In a first variant, energy deposition is achieved by focusing a laser for both propulsion and LIFT transfer modes.

In a second variant, energy is deposited via an electric field for propulsion mode.

Creating a cavitation bubble with an electric field involves using electrolysis to generate gas bubbles in a liquid. Cavitation is the physical phenomenon where gas bubbles form and implode in a liquid under variable pressure, creating forces and shock waves. The application of an electric field can facilitate the formation of these bubbles. To create a cavitation bubble with an electric field, it is known to the skilled person to use an electrolytic cell containing the conductive liquid and fitted with two electrodes connected to a source of direct current.

In a third variant, energy deposition is achieved by focusing an acoustic wave for the propulsion mode.

An acoustic wave is produced by an ultrasonic transducer designed in order to focus acoustic energy at a precise point and generate low-pressure zones in a liquid, causing gas bubbles to form.

In a particular embodiment, the receiving substrate includes damping means along the object transfer axis.

According to a variant, the transfer of biological objects is carried out without contact by generating a cavitation bubble in the liquid present between the object and the bottom of the well, the transfer being ensured:
either by converting the deposited energy into kinetic energy for the object, which falls off and leaves the liquid layer with high directivity in the propulsion mode;
or by converting the deposited energy into a movement of the liquid, which carries the objects away via a jet in LIFT mode.

Non-contact transfer ensures no plastic deformation of the object.

Advantageously, the objects to be transferred are chosen from the category of cell aggregates, spheroids, organoids, explants (Langherans islets), cell-encapsulating polymer particles (organoid covered with a layer of biomaterial), cell-seeded microcarriers, biomaterial beads.

According to a variant, the transfer is repeated to manufacture a material, tissue or organ and the volume fraction or density of the biological objects transferred by propulsion in the printed material, tissue or organ is greater than 30%.

In another variant, the transfer is carried out only once, in order to precisely characterize a single object.

In another variant, the process is combined with other printing technologies such as extrusion, inkjet, LIFT to produce complex materials or tissues with different components.

The present disclosure also relates to equipment for energy handling and transfer by deposition:
an energy source directed toward the material to be transferred,
at least one well from which the material is handled and transferred,
a target receiving substrate that collects the transferred material.

The donor consists of a plate comprising wells containing transferable objects arranged in a liquid with an orientation Dₓ, D_{y} in the plane of the liquid film and D_{z} perpendicular to the film, wherein the transfer takes place:
either by LIFT if the ratio of the size (D_{z}) of the object to the thickness (e) of the liquid film is less than 1;
or by propulsion if the ratio of the object's dimension (D_{z}) to the thickness (e) of the liquid film is greater than 1.

In one variant, energy deposition is achieved by focusing a laser for both propulsion and LIFT transfer modes.

Advantageously, the plate substrate containing the wells is transparent or weakly absorbent at the wavelength of the laser beam.

In one variant, a scanner is used to position the laser beam precisely on the centroid or center of mass of each object, thus ensuring high directivity in the transfer of the object.

In another variant, energy is deposited via an electric field.

In another variant, energy deposition is achieved by focusing an acoustic wave.

According to another variant, it includes a system for controlling and closed-loop regulating the amount of energy deposited in order to optimally transfer the object according to its size.

In another variant, it incorporates means for automating the movement of the substrate (30).

In another variant, the well plate substrate is covered by a sacrificial layer with high absorption/conduction properties for laser or electric field energy deposition.

Advantageously, it includes means for controlling a temporal succession of several energy deposits on the same well for transferring an object.

According to another variant, it includes means for controlling several spatially separated energy deposits in order to transfer several objects arranged in different wells in parallel.

According to another variant, it also comprises at least one other printing technology including extrusion, inkjet, LIFT for the manufacture of complex materials or tissues comprising different components.

According to another variant, it incorporates a system for delivering several laser beams simultaneously to the object when the latter has a non-isotropic shape, in order to guarantee its transfer along a homogeneous trajectory.

### BRIEF DESCRIPTION OF THE DRAWINGS

The present disclosure will be better understood on reading the following description, which concerns a non-limiting exemplary embodiment that is shown by the appended drawings, in which:

FIG. 1 shows a cross-sectional view of a micro-well of an example donor substrate with a small particle;

FIG. 2 shows a cross-sectional view of a micro-well of an example donor substrate with a large particle;

FIG. 3 shows a schematic view of the transfer system;

FIG. 4 shows a cross-sectional view of a micro-well variant; and

FIG. 5 shows a partial top view of a donor substrate.

### DETAILED DESCRIPTION

The present disclosure particularly concerns the additive manufacturing of a biological graft by transferring biological objects with dimensions greater than 100 μm, such as spheroids or organoids, onto a target surface. Spheroids are three-dimensional (3D) cellular aggregates that can mimic tissues. When seeded in a well of a microplate with a pyramidal or truncated cone base, these aggregates form a discrete spheroid.

Spheroids contain both deeply embedded cells and those with exposed surfaces, proliferating and non-proliferating cells, and a center surrounded by a well-oxygenated outer layer of cells. Their assembly by transfer onto a target substrate enables the creation of three-dimensional tissues such as cartilage, for example, to reconstitute damaged cartilage.

Spheroids and organoids can be made up of different stem, progenitor and/or differentiated cells, e.g., cardiac, brain, liver cells, etc.

Spheroids and organoids can be grown in single-well plates, or in 12, 24, 48, 96 or even 384-well plates containing several thousand to tens of thousands of micro-wells.

### Overview of the Donor Substrate

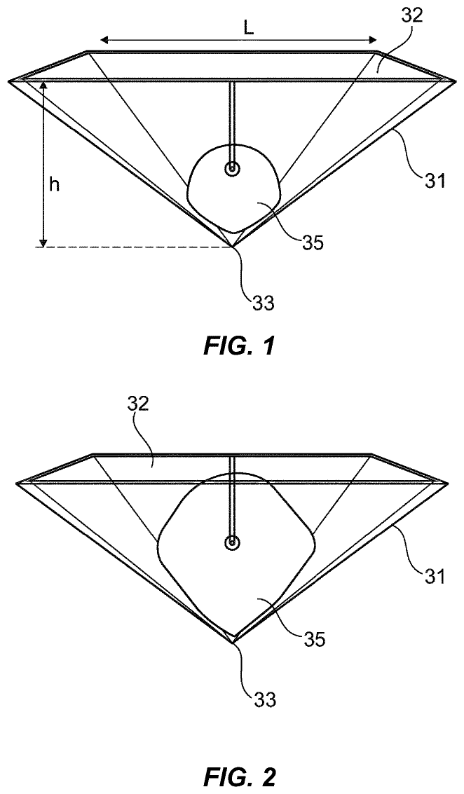

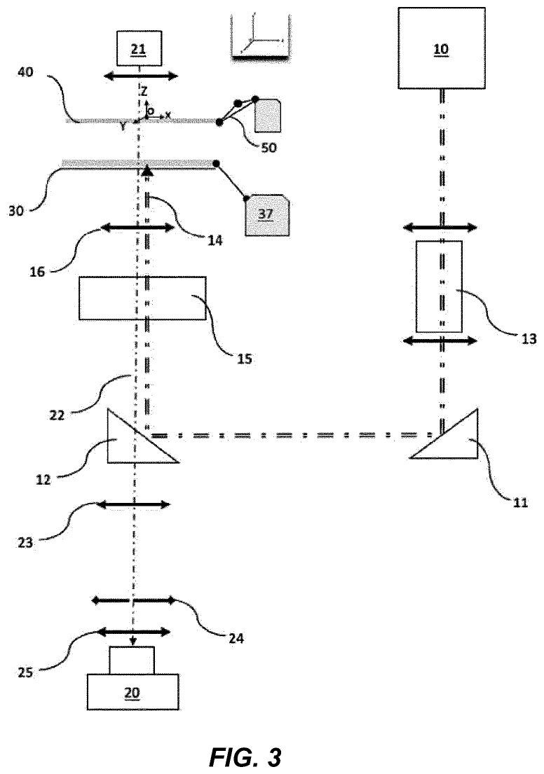

FIGS. 1 and 2 show views of a micro-well (31) on a donor substrate (30) (FIG. 3). The micro-well (31) is made from a transparent plate comprising a micro-well matrix, and has an inverted pyramidal shape with an open square base (32) of width L typically between 200 μm and 800 μm and a pointed or preferably flat bottom (33). Its height h is typically between 200 μm and 800 μm.

The spheroid (35) (FIG. 1) is made up of around 500 cells, and occupies only part of the height h of the micro-well,

The spheroid (36) (FIG. 2) is made up of around 2,000 cells, and occupies the entire part of the micro-well of height h.

An aqueous liquid, e.g., water with added salts or a culture medium, e.g., a 2% BSA (Bovine Serum Albumin) solution, at least partially fills the micro-well (31).

Depending on the size of the spheroid (35, 36) contained in the micro-well (31), the liquid either completely covers the spheroid, or the spheroid rests in a liquid base.

In the first case, transfer takes place in LIFT mode, with moderate power, typically 15 to 20 microjoules.

In the second case, the transfer is carried out by propulsion, without the particle being carried away in a liquid bubble, with a power 2 to 5 times greater, on the order of 30 to 60 microjoules.

The amount of energy deposited is minimized in order to transfer the object according to its size, while guaranteeing its post-transfer integrity. Minimizing the energy deposited also ensures a low transfer speed, enabling slow deposition on the receiving substrate, thus contributing to the integrity of the transferred object.

Optionally, the surface of the micro-well (31) is coated with a sacrificial layer, typically a layer of gold, to promote the formation of a cavitation bubble ejecting the particle contained in the micro-well. This sacrificial layer can also consist of a thin layer of metal, polymer, gel, etc.

### General Description of the Transfer System

Referring to FIG. 3, the transfer system consists of several parts:
an optical part comprising a pulsed laser (10) for generating the cavitation energy of the carrier liquid and optionally a camera (20) for observing the donor substrate (30) and the receiver substrate (40). This camera and the associated optical system are not required for shooting objects contained exclusively in micro-wells, as the positioning of the objects to be shot is constrained and does not require correction by optical observation;
an automated part, with a robot arm (50) used to automate handling of the receiver (40). The receiving substrate (40) advantageously has mechanical properties capable of absorbing shocks in order to guarantee the integrity of the transferred object, either by an elastically deformable coating or by a support mounted on a damping system along the transfer axis;
and optionally, the system can include an extruder for adding a bond, e.g., collagen, to the receiving substrate (40) between the transferred particle layers.

More generally, the equipment can combine several 3D printing, bioprinting and photopolymerization technologies.

The substrate (30) consists of a micro-well plate as described above, held by a support, which is advantageously moved by a motorized system (37), which positions the tip of one of the micro-wells in the optical axis to enable transfer of the particle contained in the micro-well with a high firing accuracy of less than 50 μm, due to knowledge of the geometry of the plate and the constrained positioning of the particle (35, 36) in the micro-well (31).

The optical part of the device is possibly made up of two parts, an optional part comprising the camera (20) for aiming at objects, and a part comprising the laser (10) for “shooting,” that is, delivering energy pulses in a plane of the donor micro-well (30) where a particle to be transferred is located.

The laser (10) is a Nd-YAG laser, for example, that emits pulses of 1 to 10 ns at 1064 nm with an energy of 15 to 60 microjoules, substantially higher than the energy normally used for LIFT processes.

Another example is a pulsed Ytterbium fiber laser emitting at 1030 nm, with shorter pulses, from 350 femtoseconds to 10 picoseconds, with several tens of microjoules of energy per pulse.

The power will be determined for each shot according to the size of the particle present in the micro-well in the optical axis, and the appropriate transfer mode.

The lens (16) is typically an F-theta lens with a focal length of 100 mm, suitable for laser scanning. Typical spot size at the focal plane is around 30 to 35 μm in diameter.

The laser beam (14) passes through shaping optics (13) and is then directed via a set of mirrors (11, 12) to a scanner (15), which then sends the beam vertically to the donor substrate (30), via the F-Theta lens (16).

The scanner (15) comprises two automated mirrors that redirect the beam horizontally at an angle toward the objective (16). The lens (16) will then straighten and focus the beam (14) so that it arrives perpendicular to the donor substrate (30) and focuses it. The scanner (15) mirrors control the movement of the laser beam (14) along horizontal axes on the donor substrate (30). The laser beam (14) is therefore focused on the donor substrate (30) and is steerable along the X and Y axes defining the horizontal plane.

For a donor substrate (30), the beam (14) is focused on the sacrificial layer coating the substrate surface, for example, a 20-nanometer gold layer deposited on a transparent optical window.

The second optional optical part comprising the camera (20) is the aiming part. It may be necessary to use a visualization system if the particles are smaller than the size of the micro-well used, for example, spheroids formed by an aggregation of cells with a diameter of 100 μm.

A visible light source, typically an LED (21), is placed above the cartridge, and the light beam (22) passes through the scanner (15) following the reverse path of the laser beam. It then arrives at a semi-reflective mirror (12), which lets visible light through to the camera (20), but reflects infrared light back to the laser (10). The beam then passes through a lens (23), an iris (24) and an objective (25) before arriving at the camera (20). Everything is aligned so that the laser beam (14) focuses on the center of the image retrieved by the camera (20).

This image can be used to determine the size of the particle present in the micro-well in the firing axis, and thus determine the power of the pulse required.

The present disclosure relates in a non-limiting way to the transfer of spheroids formed by an aggregate of cells cultivated in the laboratory and having the appearance of a small pearl made of cells and extracellular matrix.

In order to produce spheroids, stem, progenitor or differentiated cells are cultured by conventional methods and seeded in micro-wells to produce aggregates, which are then manipulated according to the method of the present disclosure.

As explained above, the “shooting zone imaging” section is optional. It is useful when micro-wells can accommodate small particles whose positioning is not constrained by the walls of the micro-well. It is then necessary to use the information provided by an imaging system to center the laser beam axis precisely, to within 50 μm, with the center of the particle.

On the other hand, when the particles are systematically large, larger than the cross-section at mid-height of the micro-well, this imaging subsystem is optional and can be omitted, since the accuracy of the shot results from the constrained positioning of the particle in the micro-well and the precise positioning of the substrate having the micro-wells relative to a framework of the equipment.

### Flat-Bottomed Micro-Wells

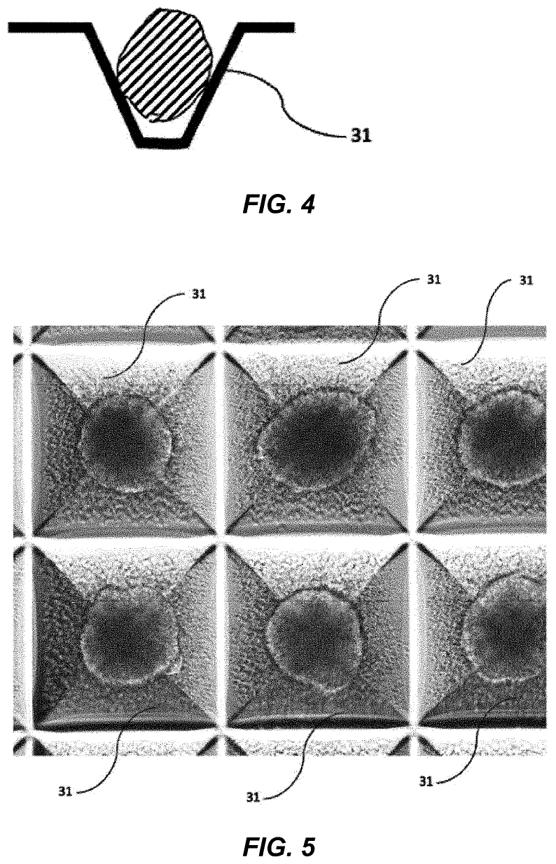

According to a variant shown in FIG. 4, the micro-wells have an inverted pyramidal cavity with a truncated tip, to limit the reflection and diffraction phenomena of the laser shot centered on the micro-well. The surface area of the micro-well base is greater than the width of the laser beam, typically between 50 and 100 μm.

1. A method of transferring an object from a donor substrate toward a target substrate, a film of carrier liquid containing the particles to be transferred being deposited on the donor substrate containing the object to be transferred, the object having dimensions (Dₓ, D_{y}, D_{z}), the method comprising:
locally exciting the liquid with energy to form a cavitation bubble localized at the object, wherein:
a. the donor substrate comprises a micro-well plate having a plurality of wells each forming a receptacle having an open upper base that tapers toward the bottom, a width (L) of the base of the wells being L>2DₓD_{y} and a height (h) of the wells (h) being h>2D_{z}, and
b. the transfer takes place
according to a first transfer mode with a first energy level E₁ if the ratio of the dimension (D_{z}) of the object to the thickness (e) of the liquid film is less than 1; and
according to a second transfer mode with a second energy level E₂>E₁ if the ratio of the dimension (D_{z}) of the object to the thickness (e) of the liquid film is greater than 1.

2. The method of claim 1, wherein the locally exciting the liquid with energy comprises focusing a laser on the liquid for a propulsion transfer mode or for a LIFT transfer mode.

3. The method of claim 1, wherein the locally exciting the liquid with energy comprises applying an electric field to the liquid for a propulsion transfer mode.

4. The method of claim 1, wherein the locally exciting the liquid with energy comprises focusing an acoustic wave on the liquid for a propulsion transfer mode.

5. The method of claim 1, wherein the target substrate comprises damping means along the object transfer axis.

6. The method of claim 1, wherein the transferring of the object is carried out without contact by the generation of the cavitation bubble in liquid present between the object and the bottom of the well, the transfer being ensured either:
by converting the deposited energy into kinetic energy for the object, which falls off and leaves the liquid layer with high directivity in the propulsion mode; or
by converting the deposited energy into a movement of the liquid, which carries the objects away via a jet in a LIFT transfer mode.

7. The method of claim 1, wherein the object to be transferred is selected from the category of cell aggregates, spheroids, organoids, explants (Langherans islets), cell-encapsulating polymer particles (organoid covered with a biomaterial layer), cell-seeded microcarriers, biomaterial beads.

8. The method of claim 1, wherein the transfer is repeated to produce a material, tissue or organ and wherein a volume fraction or volume density of the biological objects transferred by propulsion in the printed material, tissue or organ is greater than 30%.

9. The method of claim 1, wherein the transfer is carried out only once.

10. The method of claim 1, further comprising carrying out at least one additional printing technology process selected from among extrusion, inkjet, or LIFT to manufacture a material or tissue comprising different components.

11. A system for energy handling and transfer by deposition comprising:
an energy source configured to direct energy toward a material to be transferred;
at least one well in a donor substrate from which the material is handled and transferred;
a target receiving substrate that collects the transferred material;
wherein the donor substrate comprises a plate comprising the at least one well containing a transferrable object arranged in a liquid film, the transferrable object having dimensions Dₓ, D_{y} in a plane of the liquid film and a dimension D_{z} perpendicular to the liquid film, wherein
a. the donor substrate comprises a micro-well plate having a plurality of wells each forming a receptacle having an open upper base that tapers toward a bottom of each well, a width (L) of the base of the wells being L>2DₓD_{y} and the height (h) of the wells being h>2D_{z},
and wherein
b. the system is configured to transfer the transferrable object to the target receiving substrate either:
by LIFT if a ratio of the size (D_{z}) of the object to the thickness (e) of the liquid film is less than 1 with a first energy level E₁; or
by propulsion if the ratio of the size (D_{z}) of the object to the thickness (e) of the liquid film is greater than 1 with a second energy level E₂>E₁; and
wherein the system incorporates a system for delivering several laser beams simultaneously to the object when the object has a non-isotropic shape for its transfer along a homogeneous trajectory.

12. The system of claim 11, wherein the system is configured to focus a laser for two transfer modes including a propulsion transfer mode and a LIFT transfer mode.

13. The system of claim 11, wherein the donor substrate is transparent or weakly absorbing at a wavelength of a laser beam of the several laser beams.

14. The system of claim 11, further comprising a scanner enabling a laser beam of the several laser beams to be positioned precisely on a centroid or center of mass of the transferable object, thus ensuring high directivity in the transfer of the object.

15. The system of claim 11, wherein the energy source is configured to generate an electric field.

16. The system of claim 11, wherein the energy source is configured to generate an acoustic wave.

17. The system of claim 11, further comprising a system for controlling and closed-loop regulating a value of energy deposited to transfer the object as a function of a size of the object.

18. The system of claim 11, further comprising means for automating movement of the donor substrate.

19. The system of claim 11, wherein the donor substrate is covered by a sacrificial layer having laser or electric field energy deposition absorption/conduction properties.

20. The system of claim 11, further comprising means for controlling a temporal succession of several energy deposits on the same well for the transfer of an object.

21. The system of claim 11, further comprising means for controlling a plurality of spatially separated energy deposits to transfer a plurality of objects arranged in different wells in parallel.

22. The system of claim 11, further comprising a system for performing at least one other printing technology process selected from among extrusion, inkjet, and LIFT for manufacture of complex material or tissue comprising different components.
